(19) United States
(12) Patent Application Publication
(10) Pub. No.: US 2008/0194009 A1
(43) Pub. Date: Aug. 14, 2008

(54) Novel HVAC pathogen neutralization system

(76) Inventor: Rodger T. Marentis, Macungie, PA (US)

Correspondence Address: Rodger T. Marentis, 2845 Rolling Green Place, Macungie, PA, 18062, US

(21) Appl. No.: 11/705,893

(22) Filed: Feb. 13, 2007

(51) Int. Cl. C12M 1/00

(52) U.S. Cl. 435/283.1

(57) ABSTRACT

A novel process neutralizes airborne pathogens by using microwave powered ultra violet light combined with catalyzed filters and impingement media made of a layer of Filtrite filter media or comparable brand filter media covering an aerogel blanket. The geometric design maximizes the efficiency of the UV light, and impingement of pathogens on the Filtrite filter media/aerogel blanket catalyzed surfaces. The system can be utilized for preventing sick building syndrome (SBS), to prevent the spread of airborne diseases in hospitals, biohazard research laboratories, food and pharmaceutical processing facilities, and biological weapons storage facilities. It can also be used in HVAC systems to minimize the contamination caused by a biological terrorist attack.

### STATEMENT REGARDING FEDERALLY SPONSORED RESEARCH OR DEVELOPMENT

(Not applicable.)

FIELD OF THE INVENTION

The invention relates generally to apparatus and methods for the destruction of airborne pathogens such as bacteria, viruses, spores, mold and fungi within HVAC systems.

### BACKGROUND OF THE INVENTION

Airborne biological agents such as bacteria, viruses, spores, mold, and fungi in the HVAC systems of large office buildings contribute to headaches, coughs, congestion, and other symptoms (Sick Building Syndrome or “SBS”), which affects millions of workers each year. SBS is a broad term that refers to workplaces in which employees become ill from exposure to something indoors, such as chemicals used at the workplace, compounds emitted by carpets and furnishings, bacteria, mold and other microbes. Contaminants affecting Indoor Air Quality (IAQ) can enter a building from outdoor air, or they can originate from sources within the facility.

The organisms thrive and spread through the moist dark ventilation systems. SBS is especially prevalent in newer buildings that are tightly sealed in the effort to make them energy efficient.

A major cause of SBS is a buildup of bacteria and molds within the air conditioning systems cooling coils, and these toxins are eventually entrained and transported by the convective air flow into the indoor building air. These toxins can trigger violent allergic responses from the immune system. Examples of contaminants include; pollen, dust, spores, nearby industrial emissions, vehicle exhaust, smoke, pesticide treatments, cleaning agents, volatile organic compounds (VOCs) from office furnishings, communicable bacteria or viruses, and biodispersants (mold spores) derived from moisture and standing water in drip pans, cooling coils, and ductwork. The microorganisms found in typical office buildings or industrial workplaces are viruses, bacteria, and their components, such as endotoxins, fungi and their metabolic products such as mycotoxins and antigens. Health risks increase in buildings when the pathogens bacteria concentration is increased significantly in an indoor environment and these organisms or their by-products are suspended and successfully airborne towards the breathing zone by the HVAC system. Legionnaire's disease, some pneumonias, and tuberculosis are airborne infectious diseases that can be caused by airborne bacteria distributed throughout a building by an HVAC system. Endotoxins are components of the outer membrane of some bacteria. Dangerous levels of airborne endotoxins have been reported in numerous work environments, including offices and laboratories with HVAC systems without pathogen control systems. They can cause fever and malaise, changes in white blood cell counts, and respiratory and gastrointestinal problems.

Fungi are a diverse species of pathogen that exists in over 100 000 known species. Microscopic fungi include yeasts and molds. Most fungi produce spores (structures whose role is propagation) that are carried by the air. The diameter of these spores varies from approximately 1 to 60 microns. Some fungi can invade living cells and cause infectious diseases. However, several molds produce proteins or glycoproteins that are highly antigenic i.e. capable of producing an immune response and can cause, as reactions, hypersensitivity diseases or allergies in susceptible individuals. These allergy reactions include rhinitis, allergic asthma and extrinsic allergic alveolitis. Growing molds may also produce several volatile organic compounds which causes the characteristic moldy odor.

In buildings, the most important sources of antigens relating to human health are mites, cockroaches, and molds. All these organisms produce antigens, which can cause allergic asthma and allergic rhinitis. Dust mites (acarids) and their droppings that have accumulated in bedding, furniture or in places where the relative humidity and temperature are favourable, also produce antigens. Of all the hyper sensibility diseases, only hypersensitivity pneumonitis, allergic asthma, allergic rhinitis and allergic aspergillosis are known as being a result of exposure to airborne antigens.

Certain areas of hospitals require a pathogen destroying HVAC systems to prevent the spread of contagious airborne diseases and to protect patients who have immune system deficiencies. Other buildings that require pathogen destroying HVAC systems include biohazard research laboratories, food and pharmaceutical processing facilities, and biological weapons storage facilities. These facilities require a higher level of pathogen destruction than HVAC pathogen neutralization systems targeted against SBS. Finally, government buildings, post offices, executive offices for large corporations require HVAC pathogen neutralization systems to minimize the effects of a biological attack and reduce the scale of clean-up required after such an attack. These buildings require the ultimate in pathogen killing technology to accomplish an effective defense from a biological attack.

We believe that we have invented a powerful HVAC Pathogen Neutralization System which could be the first practical and inexpensive solution to all of the above problems. The invention uses microwave powered ultra violet (UV) light combined with catalyzed filters. High power microwave UV light kills pathogens <1 micron, including viruses and bacteria, which are difficult to filter. Fungi, which are larger (1-10 microns), will be neutralized utilizing catalyzed filters, which will hold the fungi for longer residence time under the UV light.

The microwave powered UV light offers many advantages over the conventional UV light used in the systems currently on the market. First, they have a high level of reliability with no fade with >24,000 hours of service, while the conventional UV light fades continuously with use and has ¼ of the service life. The microwave lights are cool running, low pressure lamps, and therefore do not foul or cause baking of residues to reduce efficacy or cause hot spots that may result in breakage of bulbs. Probably most important for a practical system, is the microwave assisted lights can turn up or turn down instantaneously as the HVAC system turns up and down with airflow. Conventional UV lights require 20 minutes to warm up, thus preventing any modulation with airflow. Finally, the microwave lights are more compact while being ten times more powerful in pathogen destroying UV light intensity than conventional UV lamps. Keep in mind that fewer lights require less space, making the UV light systems more practical in the available space of most commercial building applications.

Titanium oxide catalyst can be coated on all internal surfaces of the pathogen destruction chamber to aid and accelerate the pathogen killing reaction of the UV light on the microbes. The catalyst will also eliminate BTX compounds (benzene, toluene, and m-xylene), which contribute to SBS. All reactants under UV light and catalysis will be converted to H₂0 and CO₂ Additionally, a sacrificial filter will be utilized at the entrance to the system to trap large inert particulate compounds, and additional filter media will be employed at the filter plates.

There are five conventional technologies that control pathogens in HVAC systems:

Particulate Filtration
Filter media physically traps airborne suspended solids in a tightly woven fiber matrix. Efficiencies vary from low to high along with particle size retention. High Efficiency Particulate Filters (HEPA) may be sized to remove certain bacteria.

Electrostatic Precipitation
Another form of physical particle retention where the electrostatic precipitator employs a charged surface to “attract” oppositely charged particles. Since they ionize or “charge” the air, low levels of ozone may be produced and affect susceptible persons.

Negative Ion Generation
This process uses a similar principle to that of the electrostatic precipitator, however, the device expels ions into the atmosphere and cause suspended particles to cling to walls, floors, and other surfaces. Typically, they are mounted in a room as opposed to ductwork installation. Since it charges the air, low levels of ozone may be produced and affect susceptible persons.

Adsorption Media
This technology uses a chemisorptive media such as activated carbon to remove dissolved gases such as VOCs, ozone, smoke, and nitrogen oxides. This technology is difficult to implement due to the high AP experienced in activated carbon adsorption beds.

Ultraviolet (UV) Light Inactivation
UV has been proven in both air and water applications to inactivate bacteria and viruses to prevent them from reproducing. Typically, a either a single UV lamp, or a “bank” of UV lamps can be installed directly into the HVAC ductwork, or adjacent to cooling coils to prevent accumulated bioorganism growth.

Of the above technologies, only HEPA filtration and UV are capable of efficiently limiting microorganism distribution of pathogens into a building via an HVAC system.

A novel HVAC pathogen neutralization system has been invented that can destroy airborne biological agents such as bacteria, viruses, spores, mold, and fungi in the HVAC systems. The invention uses microwave powered ultra violet (UV) light combined with catalyzed filters. High power microwave UV light kills pathogens <1 micron, including viruses and bacteria, which are difficult to filter. Fungi, which are larger (1-10 microns), will be neutralized utilizing catalyzed impingement substrates, which will hold the fungi for longer residence time under the UV light. The system is designed to be able to turn up or turn down instantaneously as the HVAC system turns up or turns down with airflow. Additionally, the system would be able to turn up or turn down to meet higher and lower levels of pathogens in the HVAC system. A combination of catalysts (e.g. titanium oxide) will be employed that will eliminate BTX compounds, and other catalysts (e.g. PremAir), will convert ozone to oxygen to provide fresher air.

Finally, the system employs a serpentine airflow specially designed to impinge particles on the catalyzed filter media/aerogel surface of the pathogen destruction chamber and simultaneously minimizes pressure airflow pressure drop experienced with filter pathogen destruction technologies.

### BRIEF DESCRIPTION OF THE FIGURES

The five diagrams below represent five manifestations of a serpentine flow path design. The first diagram, FIG. 1, utilizes large Lexan/glass flow diversion structures in which is housed the microwave powered lights. The flow diversion structures direct the airflow towards a catalyst coated aerogel blanket surface to impinge the particles on the electrostatically charged high surface areas of the filter media/aerogel blanket.

FIG. 2 utilizes a continuous series of strategically placed baffles in the center of the duct to cut the duct into two separate chambers that maintain a serpentine flow which directs airflow for impingement on the catalyst coated electrostatically charged filter media/aerogel blanket surfaces.

FIG. 3 utilizes a discontinuous series of strategically placed baffles that allow the airflow to move and mix between the upper and lower sections. These baffles also maintain a serpentine flow which directs airflow for impingement on the catalyst coated electrostatically charged filter media/aerogel blanket surfaces.

FIG. 4 is a simplified schematic diagram of a multiple baffle serpentine flow system with UV lights mounted in the center of the duct instead of near the upper and lower walls of the duct in FIGS. 1-3.

FIG. 5 is a simplified schematic diagram of a multiple baffle serpentine flow utilizing a 90° elbow in the duct system, showing the pathogen destruction system can be adapted to space constraints within the duct.

It is readily apparent that this invention is not limited to the embodiment which has been especially described hereinabove with reference to the drawings. On the contrary, the invention extends to alternate forms. In particular, the Catalyst Coated Translucent Filter Media/Aerogel Blanket with Electrostatic Wire Mesh Support Frame (4) and Microwave Powered UV Light (3) as described herein can be modified, made larger, or smaller, lighter, or heavier with different parts substituted therefore. It is to be specifically understood that the scope of our invention is limited only by the claims.

### SUMMARY OF THE INVENTION

A novel HVAC pathogen neutralization system has been invented that can destroy airborne biological agents such as bacteria, viruses, spores, mold, and fungi and convert BTX (benzene, toluene, and m-xylene).compounds and ozone into non toxic compounds such as carbon dioxide, water, and free oxygen, in the HVAC systems. The invention uses microwave powered ultra violet (UV) light combined with catalyzed internal surfaces and filters. High power microwave UV light kills pathogens <1 micron, including viruses and bacteria, which are difficult to filter. Fungi, which are larger (1-10 microns), and spores which require the highest levels of treatment, will be neutralized utilizing catalyzed impingement substrates such as electrostatically charged filter media/aerogel blanket, which will hold these pathogens for longer residence time under the UV light. The system is designed to be able to turn up or turn down instantaneously as the HVAC system turns up or turns down with airflow. Additionally, the system would be able to turn up or turn down to meet higher and lower levels of pathogens in the HVAC system. A combination of catalysts will be employed that will eliminate BTX compounds, and other catalysts will convert ozone to oxygen to provide fresher air. An optional steam injector system could be employed at the front of the pathogen destruction chamber to enhance the destruction efficiency of the UV light by maintaining humidity levels above 40%. An optional negative ion generator may be employed at the front of the pathogen destruction chamber that would be activated under certain high pathogen concentration circumstances such as terrorist attack to negatively charge pathogen particles for more efficient adhesion on the oppositely charged aerogel blanket. Finally, the system employs a serpentine airflow specially designed to impinge pathogens and organic gases on the catalyzed aerogel surface of the pathogen destruction chamber and simultaneously minimizes airflow pressure drop experienced with HEPA filter pathogen destruction technologies.

### DETAILED DESCRIPTION

In the preferred practice of the method of the present invention, return air enters the pathogen destruction chamber (FIG. 1) through a highly porous sacrificial dust filter (1) that will minimize the pressure drop. This filter (1) will be designed so that it can be replaced easily on a frequent basis. This filter (1) represents the most frequently replaced consumable item in the pathogen destruction system. This filter (1) may or may not be catalyzed to enhance pathogen destruction of the UV light system (3) that is located just beyond the filter. This filter (1) will also have an automatic shut off device of the complete system if someone were to attempt to open the locked mechanism that is utilized to change the filter.

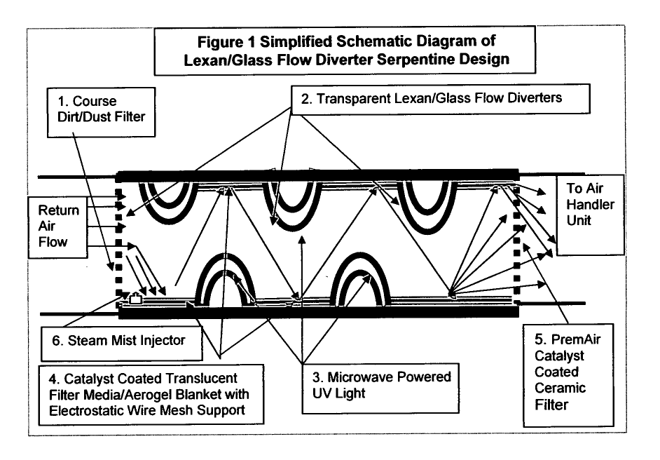

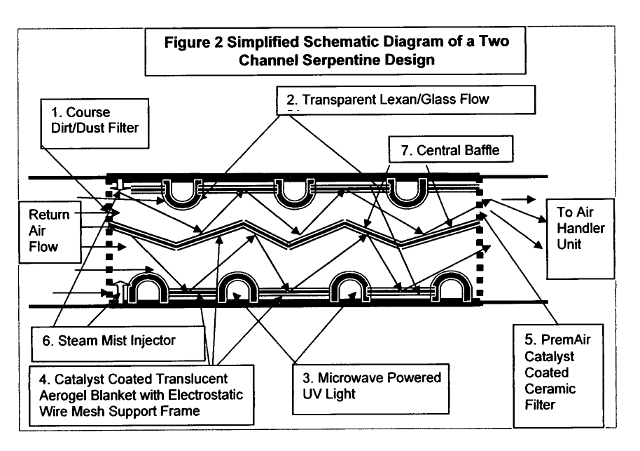

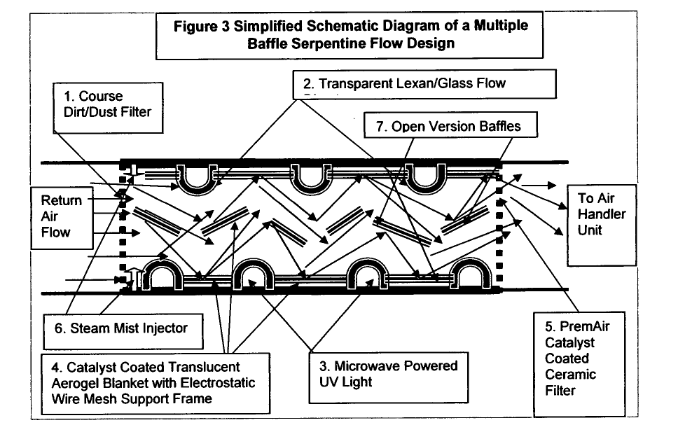

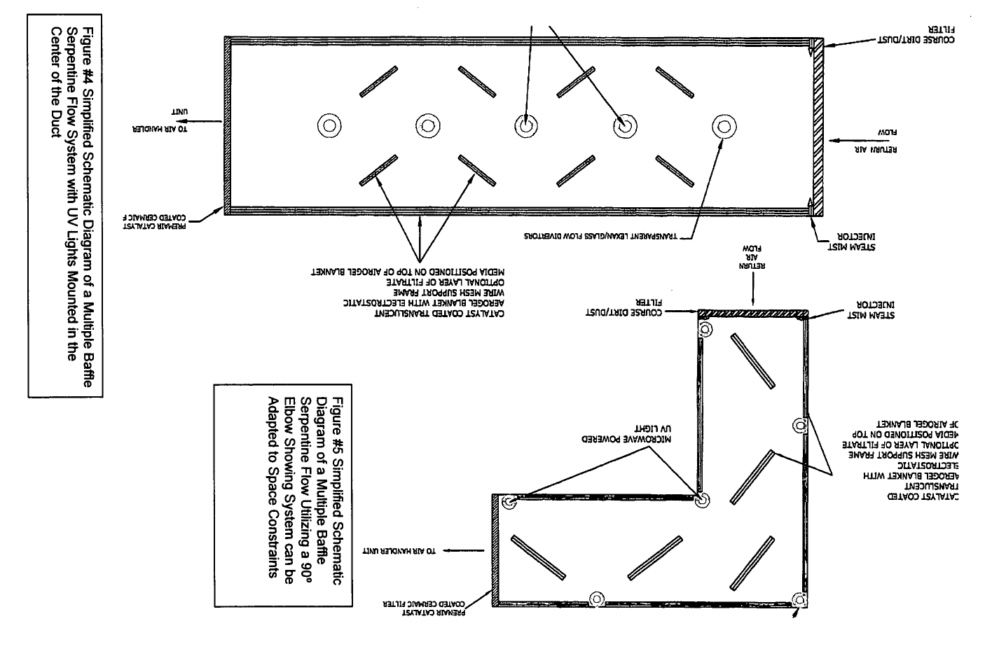

On the other side of the filter (1) is half elliptical shaped transparent Lexan or Glass flow diverter (2). This flow diverter (2) serves several functions: A) It houses and protects the microwave UV light (3). B) It diverts the horizontal flow into a more vertically angled flow that is directed to catalyst coated translucent aerogel blanket with electrostatic wire mesh support frame (4). This change in direction of flow will allow fungi and spore particles to be impinged on the high surface area of both the filter media and the aerogel component of the filter media/aerogel blanket component (4). Further enhancing the adhesion of the pathogens will be an electrostatic charge imparted on the aerogel blanket by its wire mesh support frame (4). Trapping the pathogens within the filter media/aerogel blanket is important because this will allow more time for the ultraviolet lights (3) to destroy the DNA structure of the pathogens rendering them harmless. C) The transparent Lexan/Glass flow diverters (2) will allow for areas of high intensity of UV light in passageways between the UV lights (3), especially in the middle 1/3 of the pathogen destruction chamber (FIG. 1) on an axial or horizontal plane. The high intensity of the UV light (3) in the disinfection zones between the flow diverters (2) will be powerful enough to kill nearly all biological pathogens, especially when the lights are turned to full intensity. D) Additionally, the transparent Lexan/Glass flow diverters (2) can be coated with catalyst such as titanium oxide to further enhance the pathogen destruction capabilities of the microwave powered UV lights (3). E) An optional steam injection nozzle (6) placed at the front end of the pathogen destruction chamber that will increase the humidity of the airflow to a minimum of 40% and preferably 50% in times of high pathogen loading. FIG. 2 is similar to FIG. 1 except a central baffle (7) replaces the flow diverters (2) in FIG. 1, with the function of directing airflow to impinge on the catalyst coated translucent aerogel blanket (4). FIG. 3 is a more open design version of baffles (7) but otherwise similar to FIG. 2. FIG. 4 shows an open version of baffles with the UV lights located in the center of the duct as opposed to at the top and bottom of the duct. FIG. 5 shows an open baffle system incorporated into a 90 degree elbow duct system.

Microwave powered ultra violet light has many advantages including a high level of reliability when compared with conventional UV light systems. Unlike conventional UV lights, they do not fade with tens of thousands of hours of service and generally are replaced at >24,000 hours service. They are also cool running low pressure lamps that avoid fouling or baking of residues. They operate instantly when switched on while conventional UV lights require 20 minute warm up period. Microwave powered UV lights are a more compact power supply and require fewer lamps which occupy much less space for the same light intensity than conventional UV lights. Microwave powered UV lights work by destroying the DNA structure of pathogens (bacteria, viruses, protozoa, algae, and yeast, and have ten times the power of conventional UV lamps. Microwave powered UV lights have a variable continuous power range from 50-800 watts. A 6-log bacterial reduction in 3 seconds has been achieved utilizing microwave powered UV lights due to their extraordinarily high UV light intensity. Yet, microwave powered UV lights have surprisingly high energy efficiency for germicidal UV when compared to conventional UV lights.

The filter media/aerogel blanket will be utilized on all the internal surfaces of the pathogen destruction chamber with the exception of the transparent Lexan/Glass flow diverters. The filter media/aerogel blanket will destroy biological pathogens by allowing said pathogens to be impinged in its high surface area porous structure from airflow directed at an oblique angle at its surface. Additionally, an electrostatic charge can be imparted on the filter media/aerogel blanket through its wire mesh support frame, contributing to the adsorption of pathogens onto the surface of the filter media/aerogel blanket. Furthermore, the surface of the filter media/aerogel will be coated with titanium oxide which will contribute to the destruction efficiency of the UV light within the pathogen destruction chamber. The catalysts will be titanium oxide for destruction of biological pathogens and BTX compounds.

Additionally near the end of the pathogen destruction chamber and on the porous low pressure drop filter (5) at the outlet of the pathogen destruction system, PremAir catalyst will be utilized to convert ozone molecules to oxygen to create “fresher” air in the return to the air handler unit.

The number of microwave powered lights will depend on the size of the duct and the degree and type of pathogen destruction required. Larger pathogen destruction systems required to neutralize an anthrax terrorist attack will require more microwave light modules than systems built to mitigate sick building syndrome (SBS). Additionally, some systems may require humidity control with steam injection while others with less stringent pathogen destruction requirements will not require this option. An optional negative ion generator may be employed at the front of the pathogen destruction chamber that would be activated under certain high pathogen concentration circumstances such as biological terrorist attack to negatively charge pathogen particles for more efficient adhesion on the oppositely charged aerogel blanket.

1. An apparatus of neutralizing pathogens in an HVAC system comprising directing an air stream through a single or a series of several microwave powered ultra violet (UV) lights, so that at least a portion of said micro organisms in said HVAC air stream are effectively sterilized.

2. An apparatus of neutralizing pathogens in an HVAC system set forth in claim 1, further comprising directing said air stream at a catalyzed filter/aerogel blanket media.

3. An apparatus of neutralizing pathogens in an HVAC system set forth in claim 1, that utilizes flow direction devices such as but not limited to baffles within the pathogen destruction chamber in which a serpentine pathway is provided that allows the airflow containing the microbes to impinge upon the porous surface area of the filter media/aerogel blanket.

4. An apparatus of neutralizing pathogens in an HVAC system set forth in claim 1, which utilizes a filter media on the surfaces of the pathogen destruction chamber for impingement of larger pathogens or as a filter entrapment media for smaller particles within the pathogen destruction chamber.

5. An apparatus of neutralizing pathogens in an HVAC system set forth in claim 1, which utilizes an aerogel blanket on the surfaces of the pathogen destruction chamber for impingement of larger pathogens or as a filter entrapment media for smaller particles within the pathogen destruction chamber.

6. An apparatus of neutralizing pathogens in an HVAC system set forth in claim 1 that utilizes a filter media covering an aerogel blanket on the surfaces of the pathogen destruction chamber for impingement of larger pathogens on the filter media and entrapment of smaller particles within the aerogel blanket.

7. An apparatus of neutralizing pathogens in an HVAC system set forth in claim 1 that utilizes titanium oxide catalyst coated on the filter media/aerogel blanket surface and the glass Lexan surface of the airflow diverters.

8. An apparatus of neutralizing pathogens in an HVAC system set forth in claim 1 that utilizes a serpentine pathway that forces pathogens to pass closer to the powerful microwave UV lights than the duct would allow without baffles forcing the air stream into a serpentine flow path.

9. An apparatus of neutralizing pathogens in an HVAC system set forth in claim 1 that utilizes Engelhard PremAir Catalyst or comparable catalyst to convert ozone to oxygen by coating the surface of the filter media/aerogel blanket, glass or Lexan surface of the airflow diverters, and/or sacrificial filters at the front and back end of the pathogen destruction chamber.

10. An apparatus of neutralizing pathogens in an HVAC system set forth in claim 1 that utilizes an aerogel blanket on the surfaces of the pathogen destruction chamber for impingement of larger pathogens or as a filter media within the pathogen destruction chamber and also utilizes an electrostatic charging system consisting of a grid of epoxy coated aluminum wires between the surface of the duct and the aerogel impingement blanket or the filter media or directly above the aerogel impingement blanket, or between two filter media sections at the inlet to the destruction chamber.

11. An apparatus of neutralizing pathogens in an HVAC system set forth in claim 1 that utilizes UV light and a titanium oxide coated filter media/aerogel blanket, on a large portion of the surfaces of the pathogen destruction chamber, and a system of nozzles within the chamber to spray an aerosol of water vapor/steam during times of high pathogen concentration.

12. An apparatus of neutralizing pathogens in an HVAC system set forth in claim 1 that further comprises a means for controlling the flow rate of the HVAC air stream, so that the residence time of said air stream is effective for nearly complete destruction of said micro organisms in said HVAC air stream.
